(19) United States
(12) Patent Application Publication
(10) Pub. No.: US 2009/0076341 A1
(43) Pub. Date: Mar. 19, 2009

(54) Adherent Athletic Monitor

(75) Inventors: Kristofer J. James, Eagan, MN (US); Badri Amurthur, Los Gatos, CA (US); Mark J. Bly, Falcon Heights, MN (US); Imad Libbus, Saint Paul, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corventis, Inc., San Jose, CA (US)

(21) Appl. No.: 12/209,269

(22) Filed: Sep. 12, 2008

Related U.S. Application Data
(60) Provisional application No. 60/972,336, filed on Sep. 14, 2007.
(60) Provisional application No. 60/972,537, filed on Sep. 14, 2007.
(60) Provisional application No. 60/972,343, filed on Sep. 14, 2007.
(60) Provisional application No. 61/055,666, filed on May 23, 2008.

(51) Int. Cl. A61B 5/02; G08B 23/00

(52) U.S. Cl. 600/301; 340/573.1

(57) ABSTRACT

A system is provided for tracking an individual, engaged in extreme physical activity, physiological status and detecting and predicting negative physiological events. A monitoring system is provided that includes a plurality of sensors. Each sensor has a sensor output, and a combination of the sensor outputs is used to determine distress of the monitored individual engaged in extreme physical activity. A wireless communication device is coupled to the plurality of sensors and transfers data directly or indirectly from the plurality of sensors to a remote monitoring system. A remote monitoring system is coupled to the wireless communication device and configured to receive the processed data.

### CROSS-REFERENCES TO RELATED APPLICATIONS

The present application claims the benefit under 35 USC 119(e) of U.S. Provisional Application Nos. 60/972,343, 60/972,537, 60/972,336 all filed Sep. 14, 2007, and 61/055,666 filed May 23, 2008; the full disclosures of which is incorporated herein by reference in their entirety.

The subject matter of the present application is related to the following applications: 60/972,512; 60/972,329; 60/972,354; 60/972,616; 60/972,363; 60/972,581; 60/972,629; 60/972,316; 60/972,333; 60/972,359; 60/972,340 all of which were filed on Sep. 14, 2007; 61/047,875 filed Apr. 25, 2008; 61/055,662 and 61/055,645 both filed May 23, 2008; and 61/079,746 filed Jul. 10, 2008.

The following applications are being filed concurrently with the present application, on Sep. 12, 2008: Attorney Docket Nos. 026843-000110US entitled “Multi-Sensor Patient Monitor to Detect Impending Cardiac Decompensation Prediction”; 026843-000220US entitled “Adherent Device with Multiple Physiological Sensors”; 026843-000410US entitled “Injectable Device for Physiological Monitoring”; 026843-000510US entitled “Delivery System for Injectable Physiological Monitoring System”; 026843-000620US entitled “Adherent Device for Cardiac Rhythm Management”; 026843-000710US entitled “Adherent Device for Respiratory Monitoring”; 026843-000910US entitled “Adherent Emergency Monitor”; 026843-001320US entitled “Adherent Device with Physiological Sensors”; 026843-001410US entitled “Medical Device Automatic Start-up Upon Contact to Patient Tissue”; 026843-001900US entitled “System and Methods for Wireless Body Fluid Monitoring”; 026843-002010US entitled “Adherent Cardiac Monitor with Advanced Sensing Capabilities”; 026843-002710US entitled “Dynamic Pairing of Patients to Data Collection Gateways”; 026843-003010US entitled “Adherent Multi-Sensor Device with Implantable Device Communications Capabilities”; 026843-003110US entitled “Data Collection in a Multi-Sensor Patient Monitor”; 026843-003210US entitled “Adherent Multi-Sensor Device with Empathic Monitoring”; 026843-003310US entitled “Energy Management for Adherent Patient Monitor”; and 026843-003410US entitled “Tracking and Security for Adherent Patient Monitor.”

### BACKGROUND OF THE INVENTION

1. Field of the Invention

This invention relates generally to a system for monitoring individuals engaged in physical activity, such as athletes, soldiers, fire-fighters, scuba divers, miners, wilderness adventurers and the like, and more particularly to a monitoring system that utilizes at least two sensors to determine distress of such an individual.

Throughout the world, many people are exercising in order to improve their general health and physical fitness. For the average person, however, a lack of motivation can significantly hinder their efforts. In addition, the natural tendency is to try and achieve the greatest results in the shortest possible time. When typical measurements of physical fitness and progress such as weight loss are monitored, however, expectations may not be met. The result can be a lack of motivation, which in turn leads to a cessation of exercise.

While athletes of all ages are usually able to overcome motivational hurdles, athletes often have difficulty in accurately measuring their progress. Human nature may demand instantaneous feedback for motivation and encouragement. In addition, many athletes also may not know how to train effectively for maximal improvement. For example, competitive runners may have difficulty determining whether their pace on a particular day of training is too fast or too slow. While running on a track or treadmill may allow the runner to monitor his or her speed, speed alone is often an inadequate way to monitor optimal training levels.

Currently, there are at least three methods of providing feedback to individuals engaged in a physical activity. The first, competition, can provide feedback concerning the individual's past training efforts in a particular physical activity. Competition feedback, however, may not be provided until long after the training regimen has been completed, and therefore may only allows for adjustments in subsequent training. In addition, many individuals are only interested in improving their general health and physical fitness rather than competing against others.

Another method of providing feedback to an individual engaged in a physical activity is heart rate monitoring. Heart rate monitors are known in the exercise industry and training programs have been developed based upon the data provided by these monitors. In at least some instances, an ECG-type sensor may be worn by the individual (such as in a strap which extends about the individual's chest), and heart rate (in beats per minute) is displayed on a wrist-watch type unit. While heart rate monitoring can be a useful tool, heart rate data can be difficult to interpret. In at least some instances, individuals may resort to standardized tables in order to determine target heart rate training zones. Such standardized tables, however, may only provide generalized guidelines which may or may not be appropriate for a particular individual or a particular physical activity.

A third feedback technique which may be used by individuals performing a physical activity is lactate monitoring. Lactate is a byproduct of the anaerobic metabolic process by which energy is produced in the body. The amount of lactate present in an individual's bloodstream can provide an indication of their level of exertion. While lactate monitoring can be a useful tool, it may require drawing blood samples which are analyzed by a complex, electronic device. Thus, lactate monitoring may be invasive, costly, and may be useful for a limited number of people such as some experienced athletes and their coaches, in at least some instances.

There is a need for improved systems and methods for monitoring physiological parameters of individuals engaged in extreme physical activity, such as an athlete. There is a further need for systems and methods that remotely monitor a variety of different physiological parameters of individuals engaged in extreme physical activity.

2. Description of the Background Art

Prior patents and publications that may be relevant include: U.S. Pat. Nos. 4,121,573; 4,955,381; 4,981,139; 5,080,099; 5,353,793; 5,511,553; 5,544,661; 5,558,638; 5,724,025; 5,772,586; 5,862,802; 6,047,203; 6,117,077; 6,129,744; 6,225,901; 6,385,473; 6,416,471; 6,454,707; 6,527,729; 6,527,711; 6,551,252; 6,595,927; 6,595,929; 6,605,038; 6,645,153; 6,821,249; 6,980,851; 7,020,508; 7,054,679; 7,153,262; 2003/0092975; 2005/0113703; 2005/0131288; 2006/0010090; 2006/0155183; 2006/0031102; 2006/0058593; 2006/0074283; 2006/0089679; 2006/0122474; 2006/0155183; 2006/0224051; 2006/0261598; 2006/0264730; 2007/0021678; 2007/0027388; 2007/0038038; and 2007/0142715.

### BRIEF SUMMARY OF THE INVENTION

Embodiments of the present invention provide improved devices, systems and methods for monitoring physiological parameters of a person, such as an individual person, engaged in extreme physical activity. In many embodiments, the system comprises a person measuring system, for example a person detecting system, and a remote monitoring system. The person measuring system comprises a plurality of sensors, and a wireless communication device. Each sensor can be configured to couple to the person and provide a sensor output, and the wireless communication device can be coupled to the plurality of sensors and configured to transfer person data from the plurality of sensors. The remote monitoring system can be coupled to the wireless communication device and configured to receive the data from the plurality of sensors. At least one of the measuring system or the remote monitoring system can be configured to monitor the physiological status of the person and to determine a distress of the person in response to a combination of the sensor outputs, such that physiological sensing of performance is enhanced. Distress may be determined in many ways, for example with at least one of a weighted combination change in sensor outputs, a rate of change of at least two sensor outputs as compared to a change in the sensor outputs over a longer period of time, a tiered combination of at least a first and a second sensor output, a variance from a baseline value of sensor outputs, by a look up table. The use of a plurality of sensor can allow verification, as the first sensor output may indicate a problem that can be verified by at least a second sensor output. For example, a first sensor output can be at a high value that is greater than a baseline value, and at least one of a second or a third sensor may have outputs at a high values also sufficiently greater than a baseline value so as to verify the distress. Time weighting of the outputs of at least first, second and third sensors may also be used to indicate the distress.

In a first aspect, embodiments of the present invention provide a system for monitoring a physiological status of a person. The system comprises a person measuring system and a remote monitoring system. The person measuring system comprises a plurality of sensors and a wireless communication device. Each sensor is configured to couple to the person and provide a sensor output. The wireless communication device is coupled to the plurality of sensors and configured to transfer data from the plurality of sensors. The remote monitoring system is coupled to the wireless communication device and configured to receive the data from the plurality of sensors. The remote monitoring system is positioned remote from the person. At least one of the person measuring system or the remote monitoring system is configured to monitor the physiological status of the person and to determine a distress of the person in response to a combination of the sensor outputs.

The person measuring system may configured to determine the distress. Or, the remote system may configured to determine the distress. In many embodiments, both the person measuring system and the remote system are configured to determine the distress. Each sensor may comprise circuitry configured to provide the output. The person monitoring system may comprise an adherent patch configured to adhere to the person and support the plurality of sensors and the circuitry.

In many embodiments, the plurality of sensors is configured to measure at least one of a bioimpedance, heart rate, heart rhythm, HRV, HRT, heart sounds, respiration rate, respiratory rate variability, respiratory sounds, blood pressure, activity, posture, wake/sleep, SpO2, orthopnea, temperature, heat flux or accelerometer.

In many embodiments, the wireless communication device is configured to receive instructional data from the remote monitoring system.

In many embodiments, the system may further comprise a processor system coupled to the plurality of sensors and to the wireless communication device. The processor system is configured to receive data from the plurality of sensors and generate processed monitored individual data. The processor system may comprise at least one processor located with the remote monitoring system. The person measuring system may comprise a monitoring unit and wherein the processor system comprises a processor located with the monitoring unit. The monitoring unit may comprise logic resources configured to determine the distress of the person and detect a negative physiological event of the person.

In many embodiments, the remote monitoring system may comprise logic resources configured to determine the distress of the person and to detect a negative physiological event of the person.

In many embodiments, the person measuring system comprising the plurality of sensors is configured to provided initiation, programming, measuring, storing, analyzing and communicating data of the monitored person. The remote monitoring system is configured to predict and display a physiological event of the monitored person.

In many embodiments, the plurality of sensors is configured to measure at least one of bioimpedance, heart rate, heart rhythm, HRV, HRT, heart sounds, respiration rate, respiratory rate variability, respiratory sounds, blood pressure, activity, posture, wake/sleep, SpO2, orthopnea, temperature, heat flux or accelerometer. The activity sensor may comprise at least one of a ball switch, an accelerometer, a minute ventilation sensor, a heart rate sensor, a bioimpedance noise sensor, a skin temperature sensor, a heat flux sensor, a blood pressure sensor, a muscle noise sensor or a posture sensor.

In many embodiments, the plurality of sensors is configured to switch from a first mode to a second mode, the first mode different from the second mode. The first mode and the second mode may comprise at least one of a stand alone mode for communication directly with the remote monitoring system, a communication mode for communication an implanted device, a mode for communication with a single implanted device, and a mode to coordinate different devices coupled to the plurality of sensors with different device communication protocols. The person measuring system comprising the plurality of sensors may be configured to deactivate selected sensors to reduce redundancy.

In many embodiments, the distress of the monitored person is determined in response to a weighted combination change in sensor outputs.

In many embodiments, the system may further comprise a processor system. The processor system is configured to determine distress of the monitored individual and detect a physiological event when a rate of change of at least two sensor outputs comprises an abrupt change in the sensor outputs as compared to a change in the sensor outputs over a longer period of time.

In many embodiments, the system may further comprise a processor system. The processor system is configured to determine distress of the person and a physiological event in response to a tiered combination of at least a first sensor output and a second sensor output. The processor system is further configured to verify the first sensor output with at least a second sensor output when the first sensor output indicates the that physiological event comprises a problem for the person.

In many embodiments, the remote monitoring system is configured to determine the distress of the monitored person in response to a variance from baseline values of sensor outputs. The baseline values may be defined by a look up table.

In many embodiments, the system may further comprise a processor system. The plurality of sensors comprises at least a first sensor having a first sensor output, a second sensor having a second sensor output and a third sensor having a third sensor output. The processor system is configured to combine output of each of the first sensor, the second sensor and the third sensor to determine the distress of the person. The processor system may be configured to determine the distress of the person in response to the first sensor output at a first high value greater than a baseline value and at least one of the second sensor output or the third sensor outputs at a second high value also sufficiently greater than a second baseline value to indicate the distress of the person. The processor system may be configured to determine the distress of the person in response to time weighting the output of each of the first sensor, the second and third sensor, such that the time weighting indicates a recent event that is indicative of the distress of the monitored person.

In many embodiments, the system further comprises a processor system. The processor system may be configured to track the person's physiological status with the plurality of sensors and detect and predict negative physiological events.

In many embodiments, the outputs of the plurality of sensors comprise multiple sensing vectors that include redundant vectors.

In many embodiments, the plurality of sensors comprises current delivery electrodes and sensing electrodes.

In many embodiments, the system further comprises a processor system. The processor system comprises a tangible medium, is coupled to outputs of the plurality of sensors, and is configured to calculate blended indices to monitor the person. The blended indices may comprise at least one of heart rate, respiratory rate, response to activity, heart rate divided by respiratory rate response to posture change, heart rate plus respiratory rate, heart rate divided by respiratory rate plus bioimpedance, or minute ventilation and accelerometer.

In many embodiments, the person measuring system is configured to cycle data sampling among each sensor of the plurality of sensors to minimize energy consumption of the plurality of sensors. The person measuring system may be configured to sample data at different times for each sensor of the plurality.

In many embodiments, the plurality of sensors comprises a first sensor and a second sensor. The first sensor comprises a core sensor configured to continuously monitor and determine the distress. The person measuring system is configured to verify distress with the second sensor in response to the core sensor raising a flag.

In many embodiments, at least a first portion of the sensors are used for short term tracking, and at least a second portion of the sensors are used for long term tracking, the second portion different from the first portion.

### BRIEF DESCRIPTION OF THE DRAWINGS

FIG. 1 is a block diagram illustrating one embodiment of a patient monitoring system of the present invention;

FIGS. 2A and 2B illustrate exploded view and side view of embodiments of an adherent device with sensors configured to be coupled to the skin of a patient for monitoring purposes;

FIG. 3 illustrates one embodiment of an energy management device that is coupled to the plurality of sensors of FIG. 1;

FIG. 4 illustrates one embodiment of present invention illustrating logic resources configured to receive data from the sensors and/or the processed patient for monitoring purposes, analysis and/or prediction purposes;

FIG. 5 illustrates an embodiment of the patient monitoring system of the present invention with a memory management device;

FIG. 6 illustrates an embodiment of the patient monitoring system of the present invention with an external device coupled to the sensors;

FIG. 7 illustrates an embodiment of the patient monitoring system of the present invention with a notification device;

FIG. 8 is a block diagram illustrating an embodiment of the present invention with sensor leads that convey signals from the sensors to a monitoring unit at the detecting system, or through a wireless communication device to a remote monitoring system;

FIG. 9 is a block diagram illustrating an embodiment of the present invention with a control unit at the detecting system and/or the remote monitoring system;

FIG. 10 is a block diagram illustrating an embodiment of the present invention where a control unit encodes patient data and transmits it to a wireless network storage unit at the remote monitoring system;

FIG. 11 is a block diagram illustrating one embodiment of an internal structure of a main data collection station at the remote monitoring system of the present invention; and

FIG. 12 is a flow chart illustrating an embodiment of the present invention with operation steps performed by the system of the present invention in transmitting information to the main data collection station.

### DETAILED DESCRIPTION OF THE INVENTION

Embodiments of the present invention comprise an adherent multi-sensor patient monitor capable of tracking a subject's physiological status with a suite of sensors and wirelessly communicating with a remote site. The adherent device can monitor a subject's status during athletic activity and provide appropriate feedback and alerts.

An external, adherent patch can be configured to affix to the individual person's thorax, and may comprise multiple physiological sensors. The adherent patch can wirelessly communicate with a receiver, the configuration of which depends on the application.

The adherent device can be configured to monitor subjects for signs of distress. Distress can be defined by a deviation from measured baseline physiological parameter, such as a vital sign comprising, for example, at least one of pulse, respiration, body temperature, blood pressure. These deviations may include dehydration, respiratory disorders, and cardiac disorders. An adverse change in the subject's vital signs can trigger an alert, which may be delivered to the subject, to a partner, to centralized telemetry stations (in the case of a road race, such as a marathon, multiple stations may be placed along the race route), or to an emergency medical technician.

The physiological sensors of the device may include an ECG sensor, a hydration sensor, a temperature sensor, an activity sensor, and a posture sensor.

The adherent device may also include a global position system (GPS) receiver for monitoring of the subject's physical location.

The monitoring system may also include additional adherent sensor patches placed at additional anatomical locations, and in wireless communication with the primary adherent patch placed on the thorax.

In some embodiments, the adherent devices may be electronically paired to establish a buddy system. Each patch device would serve as both a transmitter and a receiver, and would allow communication between two paired patches for notification and delivery of alerts. Physiological distress in one patch device would alert the buddy patch device. Such a configuration may be used, for example, by a pair of scuba divers. Similarly, the buddy patch can be used for monitoring of soldiers or fire fighters in the field.

Many embodiments can be configured for continuous physiological monitoring of a subject during athletic activity, such as a marathon runner, triathlete, or scuba diver. The adherent patch devices can also be used for military applications and for monitoring first responders, such as fire fighters.

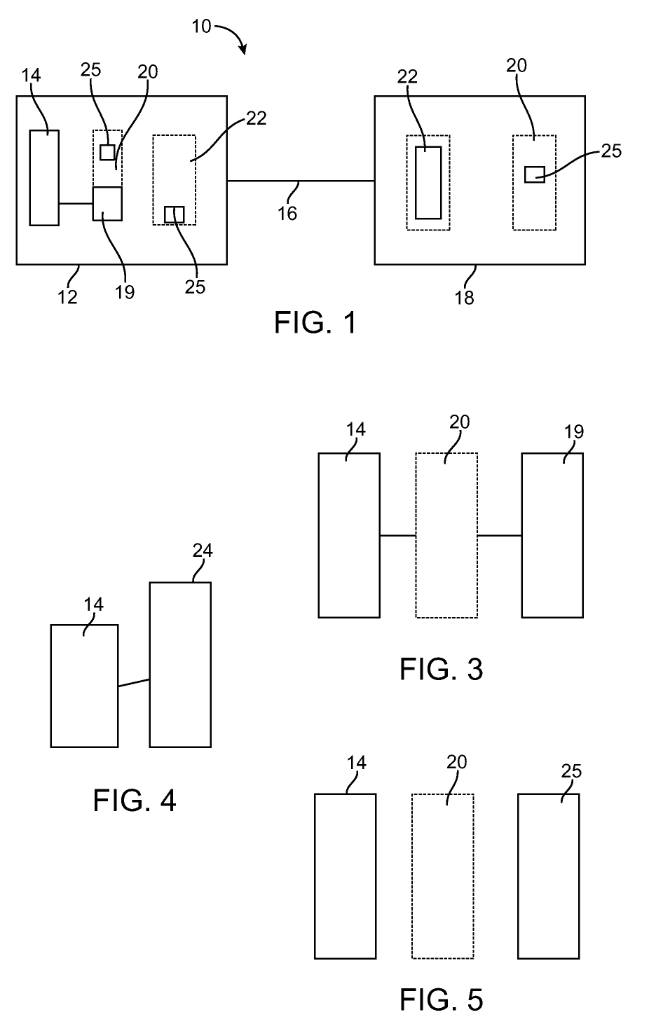

In one embodiment, illustrated in FIG. 1, the present invention is a management system, generally denoted as 10, that tracks the stress of an individual engaged in extreme physical activity, including but not limited to athletes, soldiers, firefighters, scuba divers, miners, wilderness adventurers and the like. In one embodiment, a plurality of sensors are used in combination to enhance detection and prediction capabilities as more fully explained below.

In one specific embodiment, the system 10 tracks stress level of an individual engaged in extreme physical activity. A monitoring system 12 is provided that includes a plurality of sensors 14. Each sensor 14 has a sensor output and a combination of the sensor outputs is used to determine stress of the monitored individual engaged in extreme physical activity. A wireless communication device 16 is coupled to the plurality of sensors 14 and transfers data directly or indirectly from the plurality of sensors 14 to a remote monitoring system 16. A remote monitoring system 18 is coupled to the wireless communication device 18 and is configured to receive the data. The remote monitoring system 18 is not on the individual. The detecting system 12 can continuously, or non-continuously, monitor the individual and provide alerts when required. In one embodiment, the wireless communication device 16 is a wireless local area network for receiving data from the plurality of sensors.

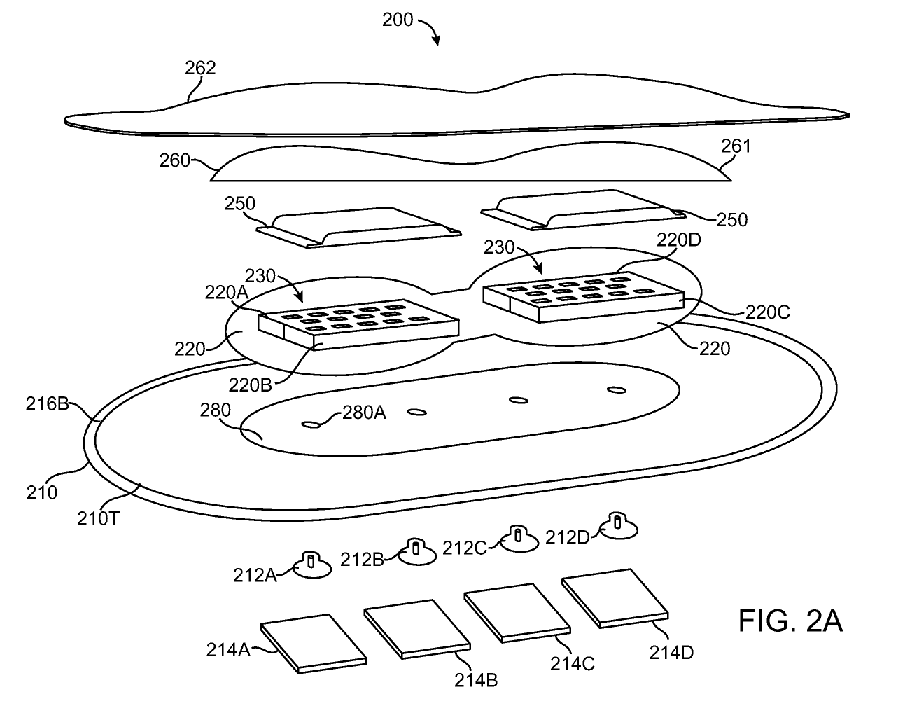

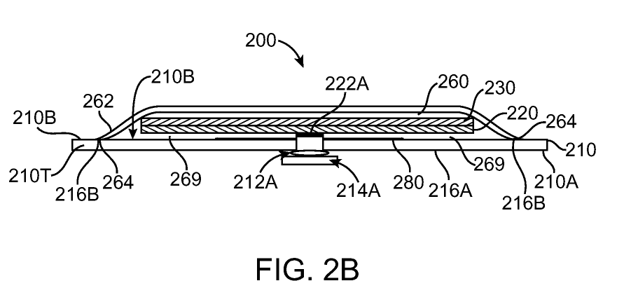

FIGS. 2A and 2B show embodiments of the plurality of sensors 14 with supported with an adherent device 200 configured to adhere to the skin. Adherent device 200 is described in U.S. App. No. 60/972,537, the full disclosure of which has been previously incorporated herein by reference. As illustrated in an exploded view of the adherent device, a cover 262, batteries 250, electronics 230, including but not limited to flex circuits and the like, an adherent tape 210T, the plurality of sensors may comprise electrodes and sensor circuitry, and hydrogels which interface the plurality of sensors 14 with the skin, are provided.

Adherent device 200 comprises a support, for example adherent patch 210, configured to adhere the device to the patient. Adherent patch 210 comprises a first side, or a lower side 210A, that is oriented toward the skin of the patient when placed on the patient. In many embodiments, adherent patch 210 comprises a tape 210T which is a material, preferably breathable, with an adhesive 216A. Patient side 210A comprises adhesive 216A to adhere the patch 210 and adherent device 200 to patient P. Electrodes 212A, 212B, 212C and 212D are affixed to adherent patch 210. In many embodiments, at least four electrodes are attached to the patch, for example six electrodes. In some embodiments the patch comprises two electrodes, for example two electrodes to measure the electrocardiogram (ECG) of the patient. Gel 214A, gel 214B, gel 214C and gel 214D can each be positioned over electrodes 212A, 212B, 212C and 212D, respectively, to provide electrical conductivity between the electrodes and the skin of the patient. In many embodiments, the electrodes can be affixed to the patch 210, for example with known methods and structures such as rivets, adhesive, stitches, etc. In many embodiments, patch 210 comprises a breathable material to permit air and/or vapor to flow to and from the surface of the skin. In some embodiments, a printed circuit board (PCB), for example flex PCB 220, may be connected to upper side 200B of patch 210 with connectors. In some embodiments, additional PCB's, for example rigid PCB's 220A, 220B, 220C and 220D, can be connected to flex PCB 220. Electronic components 230 can be connected to flex PCB 220 and/or mounted thereon. In some embodiments, electronic components 230 can be mounted on the additional PCB's.

Electronic circuitry and components 230 comprise circuitry and components to take physiologic measurements, transmit data to remote center and receive commands from remote center. In many embodiments, electronics components 230 may comprise known low power circuitry, for example complementary metal oxide semiconductor (CMOS) circuitry components. Electronics components 230 comprise an activity sensor and activity circuitry, impedance circuitry and electrocardiogram circuitry, for example ECG circuitry. In some embodiments, electronics circuitry may comprise a microphone and microphone circuitry to detect an audio signal from within the patient, and the audio signal may comprise a heart sound and/or a respiratory sound, for example an S3 heart sound and a respiratory sound with rales and/or crackles. Electronics circuitry and components 230 may comprise a temperature sensor, for example a thermistor, and temperature sensor circuitry to measure a temperature of the patient, for example a temperature of a skin of the patient.

A cover 162 can extend over the batteries, electronic components and flex printed circuit board. In many embodiments, an electronics housing 260 may be disposed under cover 262 to protect the electronic components, and in some embodiments electronics housing 260 may comprise an encapsulant over the electronic components and PCB. In some embodiments, cover 262 can be adhered to adhesive patch with an adhesive. In many embodiments, electronics housing 260 may comprise a water proof material, for example a sealant adhesive such as epoxy or silicone coated over the electronics components and/or PCB. In some embodiments, electronics housing 260 may comprise metal and/or plastic. Metal or plastic may be potted with a material such as epoxy or silicone.

Cover 262 may comprise many known biocompatible cover, casing and/or housing materials, such as elastomers, for example silicone. The elastomer may be fenestrated to improve breathability. In some embodiments, cover 262 may comprise many known breathable materials, for example polyester, polyamide, and/or elastane (Spandex). The breathable fabric may be coated to make it water resistant, waterproof, and/or to aid in wicking moisture away from the patch.

Adherent device 200 comprises several layers. Gel 214A, or gel layer, is positioned on electrode 212A to provide electrical conductivity between the electrode and the skin. Electrode 212A may comprise an electrode layer. Adhesive patch 210 may comprise a layer of breathable tape 210T, for example a known breathable tape, such as tricot-knit polyester fabric. An adhesive 216A, for example a layer of acrylate pressure sensitive adhesive, can be disposed on underside 210A of patch 210. A gel cover 280, or gel cover layer, for example a polyurethane non-woven tape, can be positioned over patch 210 comprising the breathable tape. A PCB layer, for example flex PCB 220, or flex PCB layer, can be positioned over gel cover 280 with electronic components 230 connected and/or mounted to flex PCB 220, for example mounted on flex PCB so as to comprise an electronics layer disposed on the flex PCB. In many embodiments, the adherent device may comprise a segmented inner component, for example the PCB, for limited flexibility. In many embodiments, the electronics layer may be encapsulated in electronics housing 260 which may comprise a waterproof material, for example silicone or epoxy. In many embodiments, the electrodes are connected to the PCB with a flex connection, for example trace 223A of flex PCB 220, so as to provide strain relive between the electrodes 212A, 212B, 212C and 212D and the PCB. Gel cover 280 can inhibit flow of gel 214A and liquid. In many embodiments, gel cover 280 can inhibit gel 214A from seeping through breathable tape 210T to maintain gel integrity over time. Gel cover 280 can also keep external moisture from penetrating into gel 214A. Gel cover 280 may comprise at least one aperture 280A sized to receive one of the electrodes. In many embodiments, cover 262 can encase the flex PCB and/or electronics and can be adhered to at least one of the electronics, the flex PCB or the adherent patch, so as to protect the device. In some embodiments, cover 262 attaches to adhesive patch 210 with adhesive 216B. Cover 262 can comprise many known biocompatible cover, housing and/or casing materials, for example silicone. In many embodiments, cover 262 comprises an outer polymer cover to provide smooth contour without limiting flexibility. In some embodiments, cover 262 may comprise a breathable fabric. Cover 262 may comprise many known breathable fabrics, for example breathable fabrics as described above. In some embodiments, the breathable fabric may comprise polyester, polyamide, and/or elastane (Spandex™) to allow the breathable fabric to stretch with body movement. In some embodiments, the breathable tape may contain and elute a pharmaceutical agent, such as an antibiotic, anti-inflammatory or antifungal agent, when the adherent device is placed on the patient.

In one embodiment, the wireless communication device 16 is configured to receive instructional data from the remote monitoring system 18.

The system 10 is configured to automatically detect events. The system 10 automatically detects events by at least one of, high noise states, physiological quietness, sensor continuity and compliance. In response to a detected physiological event, monitored individual states are identified when data collection is inappropriate. In response to a detected physiological event, the monitored individual states are identified when data collection is desirable. The states of the individual engaged in extreme physical activity include, physiological quietness, rest, relaxation, agitation, movement, lack of movement and a monitored individual's higher level of activity.

As illustrated in FIG. 3, an energy management device 19 is coupled to the plurality of sensors. In one embodiment, the energy management device 19 is part of the detecting system. In various embodiments, the energy management device 19 performs one or more of, modulate drive levels per sensed signal of a sensor 14, modulate a clock speed to optimize energy, watch cell voltage drop—unload cell, coulomb-meter or other battery monitor, sensor dropoff at an end of life of a battery coupled to a sensor, battery end of life dropoff to transfer data, elective replacement indicator, call center notification, sensing windows by the sensors 14 based on a monitored physiological parameter and sensing rate control.

In one embodiment, the energy management device 19 is configured to manage energy by at least one of, a thermoelectric unit, kinetics, fuel cell, through solar power, a zinc air interface, Faraday generator, internal combustion, nuclear power, a micro-battery and with a rechargeable device.

The system can use an intelligent combination of sensors to enhance detection and prediction capabilities, as more fully discloses in U.S. patent application Ser. No. 60/972,537, the full disclosure of which has been previously incorporated herein by reference, and as more fully explained below.

In one embodiment, the detecting system 12 communicates with the remote monitoring system 18 periodically or in response to a trigger event. In one embodiment, the wireless communication device 16 is a wireless local area network for receiving data from the plurality of sensors.

A processor 20 is coupled to the plurality of sensors 14 and can also be a part of the wireless communication device 16. The processor 20 receives data from the plurality of sensors 14 and creates processed data. In one embodiment, the processor 20 is at the remote monitoring system. In another embodiment, the processor 20 is at the detecting system 12. The processor 20 can be integral with a monitoring unit 22 that is part of the detecting system 12 or part of the remote monitoring system. The processor 20 may comprise a processor system having a plurality of processors with at least one processor located at each of the detecting system 12 and the remote monitoring system 18.

The processor 20 has program instructions for evaluating values received from the sensors 14 with respect to acceptable physiological ranges for each value received by the processor 20 and determine variances. The processor 20 can receive and store a sensed measured parameter from the sensors 14, compare the sensed measured value with a predetermined target value, determine a variance, accept and store a new predetermined target value and also store a series of questions from the remote monitoring system 18.

As illustrated in FIG. 4, logic resources 24, which may comprise a processor are provided that take the data from the sensors 14, and/or the processed data from the processor 20, to monitor physiologic status. The logic resources 24 can be at the remote monitoring system 18 or at the detecting system 12, such as in the monitoring unit 22.

In one embodiment, a memory management device 25 is provided as shown in FIG. 5. In various embodiments, the memory management device 25 performs one or more of data compression, prioritizing of sensing by a sensor 14, monitoring all or some of sensor data by all or a portion of the sensors 14, sensing by the sensors 14 in real time, noise blanking to provide that sensor data is not stored if a selected noise level is determined, low-power of battery caching and decimation of old sensor data.

The sensors 14 can provide a variety of different functions, including but not limited to, initiation, programming, measuring, storing, analyzing, communicating, predicting, and displaying of a physiological event of the individual engaged in extreme physical activity.

A wide variety of different sensors 14 can be utilized, including but not limited to, bioimpedance, heart rate, heart rhythm, HRV, HRT, heart sounds, respiration rate, respiration rate variability, respiratory sounds, SpO2, blood pressure, activity, posture, wake/sleep, orthopnea, temperature, heat flux and an accelerometer. A variety activity sensors can be utilized, including but not limited to a, ball switch, accelerometer, minute ventilation, HR, bioimpedance noise, skin temperature/heat flux, BP, muscle noise, posture and the like.

The outputs of the sensors 14 can have multiple features to enhance physiological sensing performance. These multiple features have multiple sensing vectors that can include redundant vectors. The sensors can include current delivery electrodes and sensing electrodes. Size and shape of current delivery electrodes, and the sensing electrodes, can be optimized to maximize sensing performance. The system 10 can be configured to determine an optimal sensing configuration and electronically reposition at least a portion of a sensing vector of a sensing electrode. The multiple features enhance the system's 10 ability to determine an optimal sensing configuration and electronically reposition sensing vectors. In one embodiment, the sensors 14 can be partially masked to minimize contamination of parameters sensed by the sensors 14.

The size and shape of current delivery electrodes, for bioimpedance, and sensing electrodes can be optimized to maximize sensing performance. Additionally, the outputs of the sensors 14 can be used to calculate and monitor blended indices. Examples of the blended indices include but are not limited to, heart rate (HR) or respiratory rate (RR) response to activity, HR/RR response to posture change, HR+RR, HR/RR+bioimpedance, and/or minute ventilation/accelerometer and the like.

The sensors 14 can be cycled in order to manage energy, and different sensors 14 can sample at different times. By way of illustration, and without limitation, instead of each sensor 14 being sampled at a physiologically relevant interval, e.g. every 30 seconds, one sensor 14 can be sampled at each interval, and sampling cycles between available sensors

By way of illustration, and without limitation, the sensors 14 can sample 5 seconds for every minute for ECG, once a second for an accelerometer sensor, and 10 seconds for every 5 minutes for impedance.

In one embodiment, a first sensor 14 is a core sensor 14 that continuously monitors and detects, and a second sensor 14 verifies the individual's distress in response to the core sensor 14 raising a flag. Additionally, some sensors 14 can be used for short term tracking, and other sensors 14 used for long term tracking.

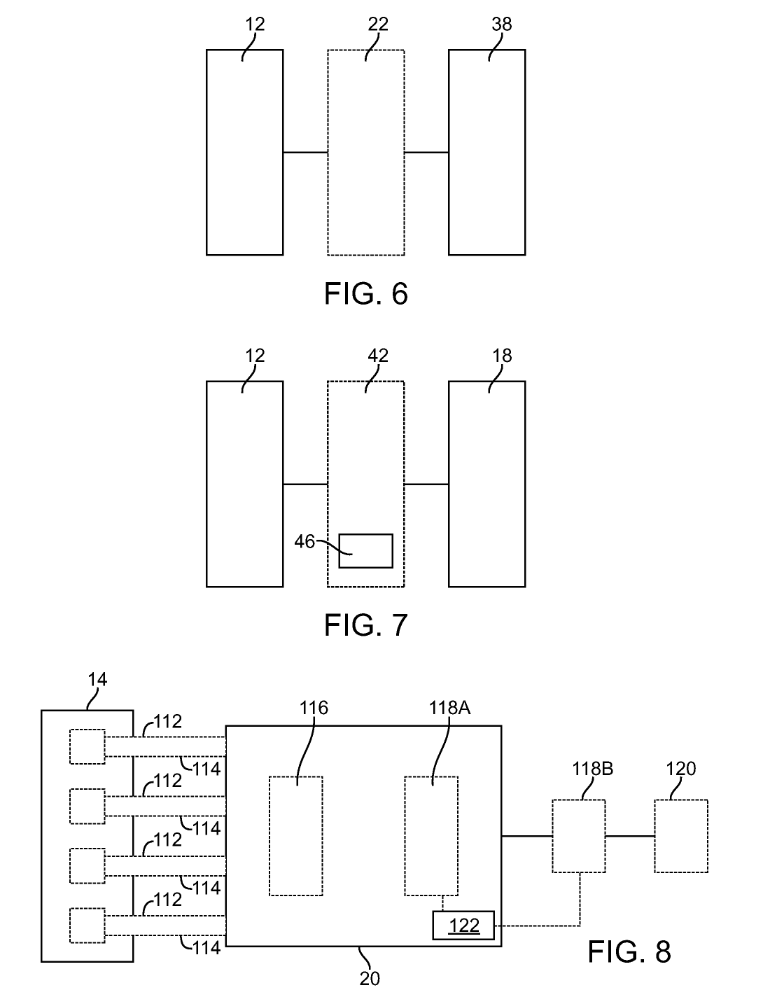

Referring to FIG. 6, in one embodiment, an external device 38 is utilized. The external device 38 can be coupled to a monitoring unit 22 that is part of the detecting system 12, or in direct communication with the sensors 14. A variety of different external devices 38 can be used.

The external device 38 can be coupled to an auxiliary input of the monitoring unit 22 at the detecting system 12 or to the monitoring system 22 at the remote monitoring system 18. Additionally, an automated reader can be coupled to an auxiliary input in order to allow a single monitoring unit 22 to be used by multiple individuals engaged in extreme physical activity. As previously mentioned above, the monitoring unit 22 can be at the remote monitoring system 18 and each monitored individual can have a monitored individual identifier (ID) including a distinct monitored individual identifier. In addition, the ID identifier can also contain monitored individual specific configuration parameters. The automated reader can scan the monitored individual identifier ID and transmit the monitored individual ID number with a monitored individual data packet such that the main data collection station can identify the monitored individual.

The sensors 14 can communicate wirelessly with the external devices 38 in a variety of ways including but not limited to, a public or proprietary communication standard and the like. In one embodiment, the sensors 14 coordinate data sharing between the external systems 38 allowing for sensor integration across devices.

In one embodiment, the processor 20 is included in the monitoring unit 22 and the external device 38 is in direct communication with the monitoring unit 22.

Referring to FIG. 7, in another embodiment, a notification device 42 is coupled to the detecting system 12 and the remote monitoring system 18. The notification device 42 is configured to provide notification when values received from the sensors 14 are not within acceptable physiological ranges. The notification device 42 can be at the remote monitoring system 18 or at the monitoring unit 22 that is part of the detecting system 12. A variety of notification devices 42 can be utilized, including but not limited to, a visible indicator, an audible alarm, an emergency medical service notification, a call center alert, direct medical provider notification and the like. The notification device 42 provides notification to a variety of different entities, including but not limited to, the individual, a caregiver, the remote monitoring system, a spouse, a family member, a medical provider, from one device to another device such as the external device 38, and the like.

Notification can be according to a preset hierarchy. By way of illustration, and without limitation, the preset hierarchy can be, the monitored individual notification first and medical provider second, monitored individual notification second and medical provider first, and the like. Upon receipt of a notification the remote monitoring system 18 can trigger a high-rate sampling of physiological parameters for alert verification.

The system 10 can also include an alarm 46, that can be coupled to the notification device 42, for generating a human perceptible signal when values received from the sensors 14 are not within acceptable physiological ranges. The alarm 46 can trigger an event to render medical assistance to the monitored individual, provide notification as set forth above, continue to monitor, wait and see, and the like.

When the values received from the sensors 14 are not within acceptable physiological ranges the notification is with the at least one of, the monitored individual, a spouse, a family member, a caregiver, a medical provider and from one device to another device, to allow for therapeutic intervention to prevent an adverse physiological event.

In another embodiment, the sensors 14 can switch between different modes, wherein the modes are selected from at least one of, a stand alone mode with communication directly with the remote monitoring system 18, coordination between different devices (external systems) coupled to the plurality of sensors and different device communication protocols.

The monitored individual's distress is determined by a weighted combination change in sensor outputs and be determined by a number of different means, including but not limited to, (i) when a rate of change of at least two sensor outputs is an abrupt change in the sensor outputs as compared to a change in the sensor outputs over a longer period of time, (ii) by a tiered combination of at least a first and a second sensor output, with the first sensor output indicating a problem that is then verified by at least a second sensor output, (iii) by a variance from a baseline value of sensor outputs, and the like. The baseline values can be defined in a look up table.

In another embodiment, the monitored individual's distress is determined using three or more sensors by at least one of, (i) when the first sensor output is at a value that is sufficiently different from a baseline value, and at least one of the second and third sensor outputs is at a value also sufficiently different from a baseline value to indicate monitored individual distress, (ii) by time weighting the outputs of the first, second and third sensors, and the time weighting indicates a recent event that is indicative of the monitored individual distress and the like.

In one embodiment, the wireless communication device 16 can include a, modem, a controller to control data supplied by the sensors 14, serial interface, LAN or equivalent network connection and a wireless transmitter. Additionally, the wireless communication device 16 can include a receiver and a transmitter for receiving data indicating the values of the physiological event detected by the plurality of sensors, and for communicating the data to the remote monitoring system 18. Further, the wireless communication device 16 can have data storage for recording the data received from the sensors 14 and an access device for enabling access to information recording in the data storage from the remote monitoring system 18.

In various embodiments, the remote monitoring system 18 can include a, receiver, a transmitter and a display for displaying data representative of values of the one physiological event detected by the sensors 14. The remote monitoring system can also include a, data storage mechanism that has acceptable ranges for physiological values stored therein, a comparator for comparing the data received from the monitoring system 12 with the acceptable ranges stored in the data storage device and a portable computer. The remote monitoring system 18 can be a portable unit with a display screen and a data entry device for communicating with the wireless communication device 16.

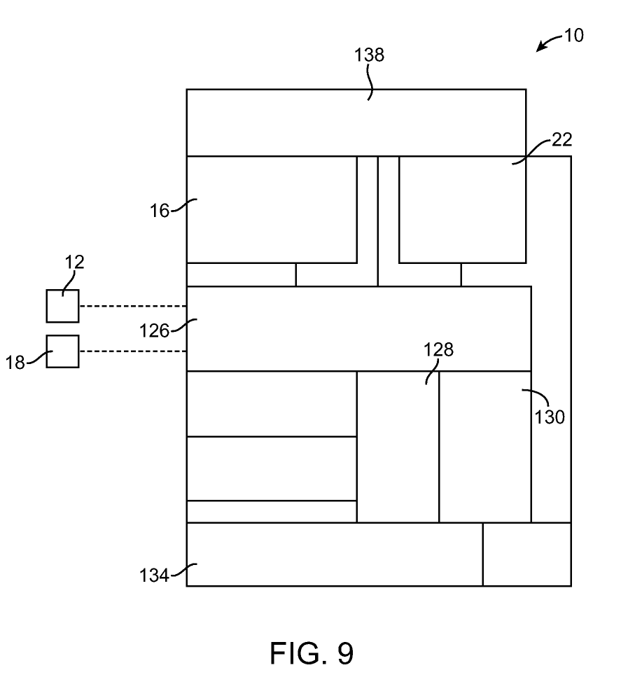

Referring now to FIG. 8, for each sensor 14, a sensor lead 112 and 114 conveys signals from the sensor 14 to the monitoring unit 22 at the detecting system 12, or through the wireless communication device 16 to the remote monitoring system 18. In one embodiment, each signal from a sensor 14 is first passed through a low-pass filter 116, at the detecting system 12 or at the remote monitoring system 18, to smooth the signal and reduce noise. The signal is then transmitted to an analog-to-digital converter 118, which transforms the signals into a stream of digital data values, that can be stored in a digital memory 118. From the digital memory 118, data values are transmitted to a data bus 120, along which they are transmitted to other components of the circuitry to be processed and archived. From the data bus 120, the digital data can be stored in a non-volatile data archive memory. The digital data can be transferred via the data bus 120 to the processor 20, which processes the data based in part on algorithms and other data stored in a non-volatile program memory.

The detecting system 12 can also include a power management module 122 configured to power down certain components of the system, including but not limited to, the analog-to-digital converters 118 and 124, digital memories 118 and the non-volatile data archive memory and the like, between times when these components are in use. This helps to conserve battery power and thereby extend the useful life. Other circuitry and signaling modes may be devised by one skilled in the art.

As can be seen in FIG. 9, a control unit 126 is included at the detecting system 12, the remote monitoring system 18 or at both locations.

In one embodiment, the control unit 126 can be a 486 microprocessor. The control unit 126 can be coupled to the sensors 14 directly at the detecting system 12, indirectly at the detecting system 12 or indirectly at the remote monitoring system 18. Additionally the control unit 126 can be coupled to a, blood pressure monitor, cardiac rhythm management device, scale, a device that dispenses medication that can indicate the medication has been dispensed.

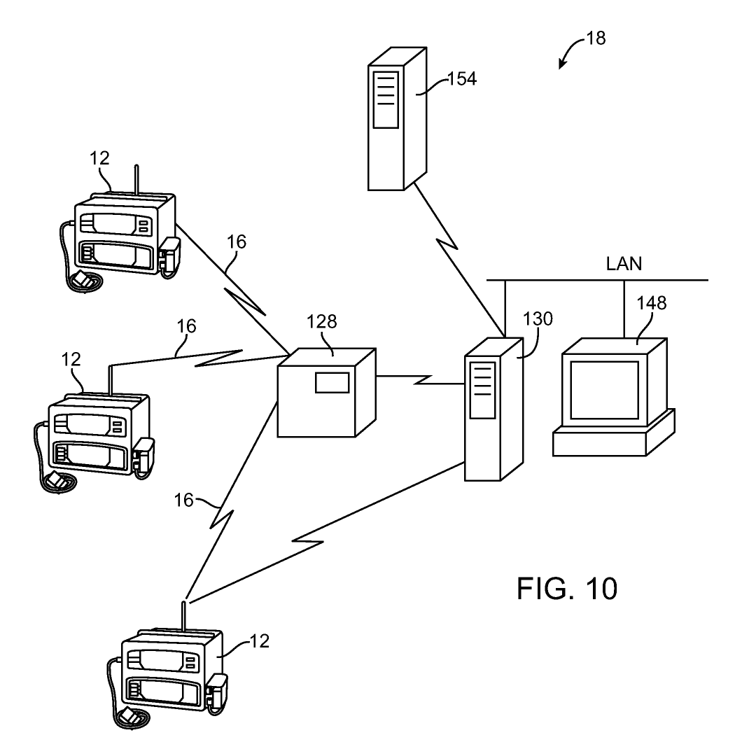

The control unit 126 can be powered by AC inputs which are coupled to internal AC/DC converters 134 that generate multiple DC voltage levels. After the control unit 126 has collected the monitored individual data from the sensors 14, the control unit 126 encodes the recorded monitored individual data and transmits the monitored individual data through the wireless communication device 16 to transmit the encoded monitored individual data to a wireless network storage unit 128 at the remote monitoring system 18 as shown in FIG. 10. In another embodiment, wireless communication device 16 transmits the monitored individual data from the sensors 14 to the control unit 126 when it is at the remote monitoring system 18.

Every time the control unit 126 plans to transmit monitored individual data to a main data collection station 130, located at the remote monitoring system 18, the control unit 126 attempts to establish a communication link. The communication link can be wireless, wired, or a combination of wireless and wired for redundancy, e.g., the wired link checks to see if a wireless communication can be established. If the wireless communication link 16 is available, the control unit 126 transmits the encoded monitored individual data through the wireless communication device 16. However, if the wireless communication device 16 is not available for any reason, the control unit 126 waits and tries again until a link is established.

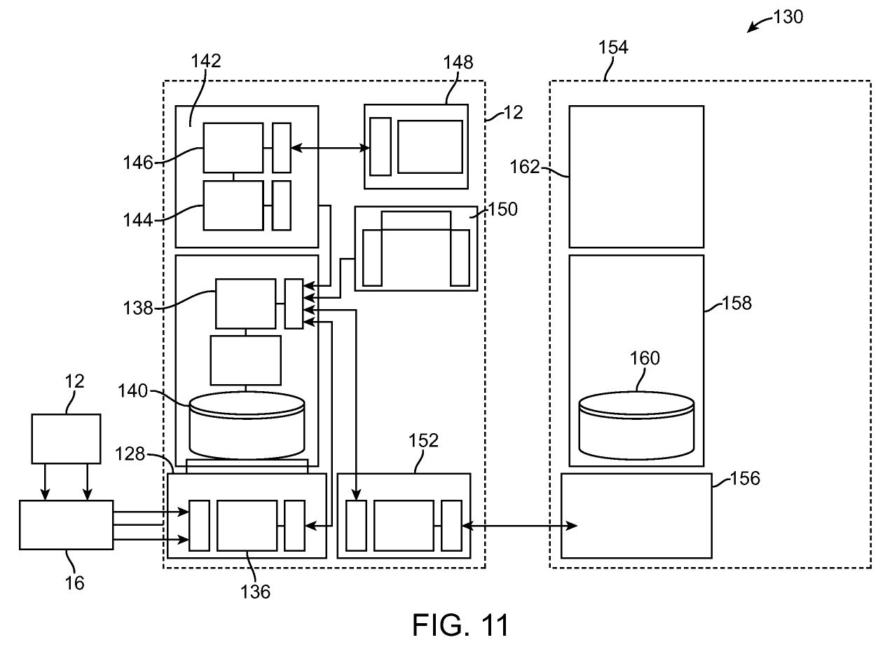

Referring now to FIG. 11, one embodiment of an internal structure of a main data collection station 130, at the remote monitoring system 18, is illustrated. The monitored individual data can be transmitted by the remote monitoring system 18 by either the wireless communication device 16 or conventional modem to the wireless network storage unit 128. After receiving the monitored individual data, the wireless network storage unit 128 can be accessed by the main data collection station 130. The main data collection station 130 allows the remote monitoring system 18 to monitor the monitored individual data of numerous monitored individuals from a centralized location without requiring the monitored individual or a medical provider to physically interact with each other.

The main data collection station 130 can include a communications server 136 that communicates with the wireless network storage unit 128. The wireless network storage unit 128 can be a centralized computer server that includes a unique, password protected mailbox assigned to and accessible by the main data collection station 130. The main data collection station 130 contacts the wireless network storage unit 128 and downloads the monitored individual data stored in a mailbox assigned to the main data collection station 130.

Once the communications server 136 has formed a link with the wireless network storage unit 128, and has downloaded the monitored individual data, the monitored individual data can be transferred to a database server 138. The database server 138 includes a monitored individual database 140 that records and stores the monitored individual data of the monitored individuals based upon identification included in the data packets sent by each of the monitoring units 22. For example, each data packet can include an identifier.

Each data packet transferred from the remote monitoring system 18 to the main data collection station 130 does not have to include any monitored individual identifiable information. Instead, the data packet can include the serial number assigned to the specific detecting system 12. The serial number associated with the detecting system 12 can then be correlated to a specific monitored individual by using information stored on the monitored individual database 138. In this manner, the data packets transferred through the wireless network storage unit 128 do not include any monitored individual-specific identification. Therefore, if the data packets are intercepted or improperly routed, monitored individual confidentiality can not be breached.

The database server 138 can be accessible by an application server 142. The application server 142 can include a data adapter 144 that formats the monitored individual data information into a form that can be viewed over a conventional web-based connection. The transformed data from the data adapter 144 can be accessible by propriety application software through a web server-146 such that the data can be viewed by a workstation 148. The workstation 148 can be a conventional personal computer that can access the monitored individual data using proprietary software applications through, for example, HTTP protocol, and the like.

The main data collection station further can include an escalation server 150 that communicates with the database server 138. The escalation server 150 monitors the monitored individual data packets that are received by the database server 138 from the monitoring unit 22. Specifically, the escalation server 150 can periodically poll the database server 138 for unacknowledged monitored individual data packets. The monitored individual data packets are sent to the remote monitoring system 18 where the processing of monitored individual data occurs. The remote monitoring system 18 communicates with a medical provider if the event that an alert is required. If data packets are not acknowledged by the remote monitoring system 18. The escalation server 150 can be programmed to automatically deliver alerts to a specific medical provider if an alarm message has not been acknowledged within a selected time period after receipt of the data packet.

The escalation server 150 can be configured to generate the notification message to different people by different modes of communication after different delay periods and during different time periods.

The main data collection station 130 can include a batch server 152 connected to the database server 138. The batch server 152 allows an administration server 154 to have access to the monitored individual data stored in the monitored individual database 140. The administration server allows for centralized management of monitored individual information and monitored individual classifications.

The administration server 154 can include a batch server 156 that communicates with the batch server 152 and provides the downloaded data to a data warehouse server 158. The data warehouse server 158 can include a large database 160 that records and stores the monitored individual data.

The administration server 154 can further include an application server 162 and a maintenance workstation 164 that allow personnel from an administrator to access and monitor the data stored in the database 160.

The data packet utilized in the transmission of the monitored individual data can be a variable length ASCII character packet, or any generic data formats, in which the various monitored individual data measurements are placed in a specific sequence with the specific readings separated by commas. The control unit 126 can convert the readings from each sensor 14 into a standardized sequence that forms part of the monitored individual data packet. In this manner, the control unit 126 can be programmed to convert the monitored individual data readings from the sensors 14 into a standardized data packet that can be interpreted and displayed by the main data collection station 130 at the remote monitoring system 18.

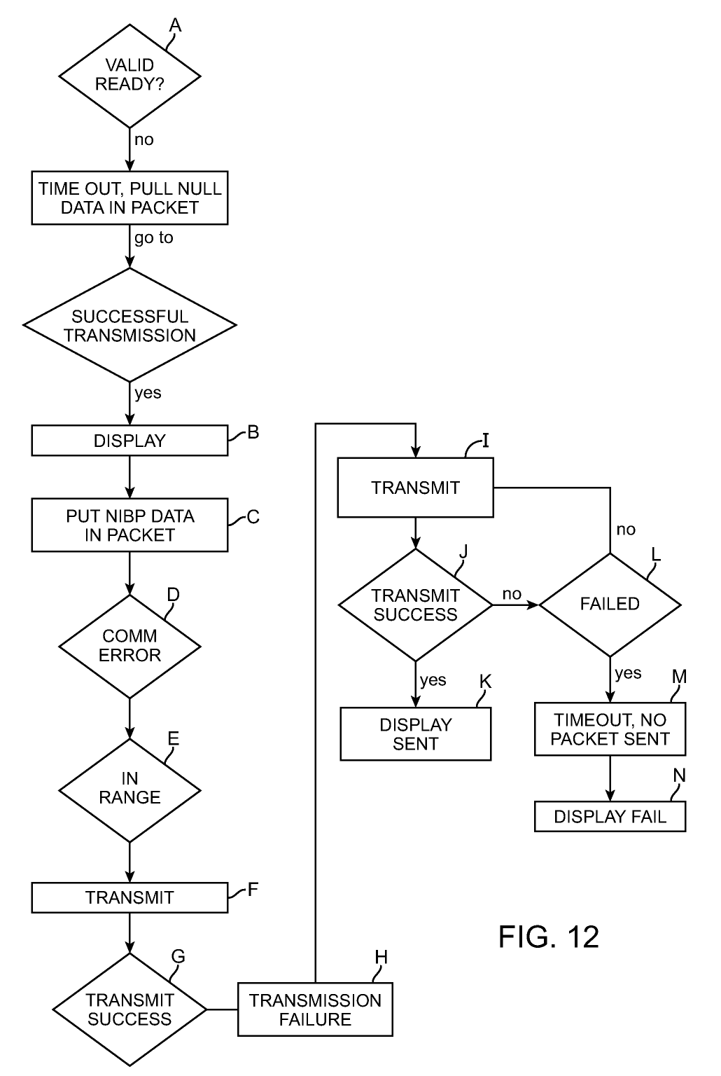

Referring now to the flow chart of FIG. 12, if an external device 38 fails to generate a valid reading, as illustrated in step A, the control unit 126 fills the portion of the monitored individual data packet associated with the external device 38 with a null indicator. The null indicator can be the lack of any characters between commas in the monitored individual data packet. The lack of characters in the monitored individual data packet can indicate that the monitored individual was not available for the monitored individual data recording. The null indicator in the monitored individual data packet can be interpreted by the main data collection station 130 at the remote monitoring system 18 as a failed attempt to record the monitored individual data due to the unavailability of the monitored individual, a malfunction in one or more of the sensors 14, or a malfunction in one of the external devices 38. The null indicator received by the main data collection station 130 can indicate that the transmission from the detecting system 12 to the remote monitoring system 18 was successful. In one embodiment, the integrity of the data packet received by the main data collection station 130 can be determined using a cyclic redundancy code, CRC-16, check sum algorithm. The check sum algorithm can be applied to the data when the message can be sent and then again to the received message.

After the monitored individual data measurements are complete, the control unit 126 displays the sensor data, including but not limited to blood pressure cuff data and the like, as illustrated by step B. In addition to displaying this data, the monitored individual data can be placed in the monitored individual data packet, as illustrated in step C.

As previously described, the system 10 can take additional measurements utilizing one or more auxiliary or external devices 38 such as those mentioned previously. Since the monitored individual data packet has a variable length, the auxiliary device monitored individual information can be added to the monitored individual data packet being compiled by the remote monitoring unit 22 during monitored individual data acquisition period being described. Data from the external devices 38 is transmitted by the wireless communication device 16 to the remote monitoring system 18 and can be included in the monitored individual data packet.

If the remote monitoring system 18 can be set in either the auto mode or the wireless only mode, the remote monitoring unit 22 can first determine if there can be an internal communication error, as illustrated in step D.

A no communication error can be noted as illustrated in step E. If a communication error is noted the control unit 126 can proceed to wireless communication device 16 or to a conventional modem transmission sequence, as will be described below. However, if the communication device is working the control unit 126 can transmit the monitored individual data information over the wireless network 16, as illustrated in step F. After the communication device has transmitted the data packet, the control unit 126 determines whether the transmission was successful, as illustrated in step G. If the transmission has been unsuccessful only once, the control unit 126 retries the transmission. However, if the communication device has failed twice, as illustrated in step H, the control unit 126 proceeds to the conventional modem process if the remote monitoring unit 22 was configured in an auto mode.

When the control unit 126 is at the detecting system 12, and the control unit 126 transmits the monitored individual data over the wireless communication device 16, as illustrated in step I, if the transmission has been successful, the display of the remote monitoring unit 22 can display a successful message, as illustrated in step I. However, if the control unit 126 determines in step K that the communication of monitored individual data has failed, the control unit 126 repeats the transmission until the control unit 126 either successfully completes the transmission or determines that the transmission has failed a selected number of times, as illustrated in step L. The control unit 126 can time out the and a failure message can be displayed, as illustrated in steps M and N. Once the transmission sequence has either failed or successfully transmitted the data to the main data collection station, the control unit 126 returns to a start program step 0.

As discussed previously, the monitored individual data packets are first sent and stored in the wireless network storage unit 128. From there, the monitored individual data packets are downloaded into the main data collection station 130. The main data collection station 130 decodes the encoded monitored individual data packets and records the monitored individual data in the monitored individual database 140. The monitored individual database 140 can be divided into individual storage locations for each monitored individual such that the main data collection station 130 can store and compile monitored individual data information from a plurality of individual monitored individuals.

A report on the monitored individual's status can be accessed by a medical provider through a medical provider workstation that is coupled to the remote monitoring system 18. Unauthorized access to the monitored individual database can be prevented by individual medical provider usernames and passwords to provide additional security for the monitored individual's recorded monitored individual data.

The main data collection station 130 and the series of work stations 148 allow the remote monitoring system 18 to monitor the daily monitored individual data measurements taken by a plurality of monitored individuals reporting monitored individual data to the single main data collection station 130. The main data collection station 130 can be configured to display multiple monitored individuals on the display of the workstations 148. The internal programming for the main data collection station 130 can operate such that the monitored individuals are placed in a sequential top-to-bottom order based upon whether or not the monitored individual can be generating an alarm signal for one of the monitored individual data being monitored. For example, if one of the monitored individuals monitored by monitoring system 130 has a blood pressure exceeding a predetermined maximum amount, this monitored individual can be moved toward the top of the list of monitored individuals and the monitored individual's name and/or monitored individual data can be highlighted such that the medical personnel can quickly identify those monitored individuals who may be in need of medical assistance. By way of illustration, and without limitation, the following paragraphs is a representative order ranking method for determining the order which the monitored individuals are displayed:

Alarm Display Order Monitored individual Status is then sorted 1 Medical Alarm Most alarms violated to least alarms violated, then oldest to newest 2 Missing Data Alarm Oldest to newest 3 Late Oldest to newest 4 Reviewed Medical Alarms Oldest to newest 5 Reviewed Missing Data Oldest to newest Alarms 6 Reviewed Null Oldest to newest 7 NDR Oldest to newest 8 Reviewed NDR Oldest to newest.

Alarm Display Order Monitored individual Status can then sorted 1 Medical Alarm Most alarms violated to least alarms violated, then oldest to newest 2 Missing Data Alarm Oldest to newest 3 Late Oldest to newest 4 Reviewed Medical Alarms Oldest to newest 5 Reviewed Missing Data Oldest to newest Alarms 6 Reviewed Null Oldest to newest 7 NDR Oldest to newest 8 Reviewed NDR Oldest to newest.

As listed in the above, the order of monitored individuals listed on the display can be ranked based upon the seriousness and number of alarms that are registered based upon the latest monitored individual data information. For example, if the blood pressure of a single monitored individual exceeds the tolerance level and the monitored individual's heart rate also exceeds the maximum level, this monitored individual will be placed above a monitored individual who only has one alarm condition. In this manner, the medical provider can quickly determine which monitored individual most urgently needs medical attention by simply identifying the monitored individual's name at the top of the monitored individual list. The order which the monitored individuals are displayed can be configurable by the remote monitoring system 18 depending on various preferences.

As discussed previously, the escalation server 150 automatically generates a notification message to a specified medical provider for unacknowledged data packets based on user specified parameters.

In addition to displaying the current monitored individual data for the numerous monitored individuals being monitored, the software of the main data collection station 130 allows the medical provider to trend the monitored individual data over a number of prior measurements in order to monitor the progress of a particular monitored individual. In addition, the software allows the medical provider to determine whether or not a monitored individual has been successful in recording their monitored individual data as well as monitor the questions being asked by the remote monitoring unit 22.

As previously mentioned, the system 10 uses an intelligent combination of sensors to enhance detection and prediction capabilities. Electrocardiogram circuitry can be coupled to the sensors 14, or electrodes, to measure an electrocardiogram signal of the monitored individual. An accelerometer can be mechanically coupled, for example adhered or affixed, to the sensors 14, adherent patch and the like, to generate an accelerometer signal in response to at least one of an activity or a position of the monitored individual. The accelerometer signals improve monitored individual diagnosis, and can be especially useful when used with other signals, such as electrocardiogram signals and impedance signals, including but not limited to, hydration respiration, and the like. Mechanically coupling the accelerometer to the sensors 14, electrodes, for measuring impedance, hydration and the like can improve the quality and/or usefulness of the impedance and/or electrocardiogram signals. By way of illustration, and without limitation, mechanical coupling of the accelerometer to the sensors 14, electrodes, and to the skin of the monitored individual can improve the reliability, quality and/or accuracy of the accelerometer measurements, as the sensor 14, electrode, signals can indicate the quality of mechanical coupling of the patch to the monitored individual so as to indicate that the device is connected to the monitored individual and that the accelerometer signals are valid. Other examples of sensor interaction include but are not limited to, (i) orthopnea measurement where the breathing rate is correlated with posture during sleep, and detection of orthopnea, (ii) a blended activity sensor using the respiratory rate to exclude high activity levels caused by vibration (e.g. driving on a bumpy road) rather than exercise or extreme physical activity, (iii) sharing common power, logic and memory for sensors, electrodes, and the like.

The signals from the plurality of sensors can be combined in many ways. In some embodiments, the signals may be used simultaneously to determine a patient distress, such as an impending heart failure.

In some embodiments, the signals can be combined by using the at least two of the electrocardiogram signal, the respiration signal or the activity signal to look up a value in a previously existing array.

TABLE 1
Lookup Table for ECG and Respiration Signals.

| Heart Rate/Respiration | A-B bpm | C-D bpm | E-F bpm |
|---|---|---|---|
| U-V per min | N | N | Y |
| W-X per min | N | Y | Y |
| Y-Z per min | Y | Y | Y |

Table 1 shows combination of the electrocardiogram signal with the respiration signal to look up a value in a pre-existing array. For example, at a heart rate in the range from A to B bpm and a respiration rate in the range from U to V per minute triggers a response of N. In some embodiments, the values in the table may comprise a tier or level of the response, for example four tiers. In specific embodiments, the values of the look up table can be determined in response to empirical data measured for a patient population of at least about 100 patients, for example measurements on about 1000 to 10,000 patients. The look up table shown in Table 1 illustrates the use of a look up table according to one embodiment, and one will recognize that many variables can be combined with a look up table.

In some embodiments, the table may comprise a three or more dimensional look up table, and the look up table may comprises a tier, or level, of the response, for example an alarm.

In some embodiments, the signals may be combined with at least one of adding, subtracting, multiplying, scaling or dividing the at least two of the electrocardiogram signal, the respiration signal or the activity signal. In specific embodiments, the measurement signals can be combined with positive and or negative coefficients determined in response to empirical data measured for a patient population of at least about 100 patients, for example data on about 1000 to 10,000 patients.

In some embodiments, a weighted combination may combine at least two measurement signals to generate an output value according to a formula of the general form

OUTPUT=aX+bY

where a and b comprise positive or negative coefficients determined from empirical data and X, and Z comprise measured signals for the patient, for example at least two of the electrocardiogram signal, the respiration signal or the activity signal. While two coefficients and two variables are shown, the data may be combined with multiplication and/or division. One or more of the variables may be the inverse of a measured variable.

In some embodiments, the ECG signal comprises a heart rate signal that can be divided by the activity signal. Work in relation to embodiments of the present invention suggest that an increase in heart rate with a decrease in activity can indicate an impending distress. The signals can be combined to generate an output value with an equation of the general form

OUTPUT=aX/Y+bZ

where X comprise a heart rate signal, Y comprises an activity signal and Z comprises a respiration signal, with each of the coefficients determined in response to empirical data as described above.

In some embodiments, the data may be combined with a tiered combination. While many tiered combinations can be used a tiered combination with three measurement signals can be expressed as

OUTPUT=(ΔX)+(ΔY)+(ΔZ)

where (ΔX), (ΔY), (ΔZ) may comprise change in heart rate signal from baseline, change in respiration signal from baseline and change in activity signal from baseline, and each may have a value of zero or one, based on the values of the signals. For example if the heart rate increase by 10%, (ΔX) can be assigned a value of 1. If respiration increases by 5%, (ΔY) can be assigned a value of 1. If activity decreases below 10% of a baseline value (ΔZ) can be assigned a value of 1. When the output signal is three, a flag may be set to trigger an alarm.

In some embodiments, the data may be combined with a logic gated combination. While many logic gated combinations can be used, a logic gated combination with three measurement signals can be expressed as

OUTPUT=(ΔX) AND (ΔY) AND (ΔZ)

where (ΔX), (ΔY), (ΔZ) may comprise change in heart rate signal from baseline, change in respiration signal from baseline and change in activity signal from baseline, and each may have a value of zero or one, based on the values of the signals. For example if the heart rate increase by 10%, (ΔX) can be assigned a value of 1. If respiration increases by 5%, (ΔY) can be assigned a value of 1. If activity decreases below 10% of a baseline value (ΔZ) can be assigned a value of 1. When each of (ΔX), (ΔY), (ΔZ) is one, the output signal is one, and a flag may be set to trigger an alarm. If any one of (ΔX), (ΔY) or (ΔZ) is zero, the output signal is zero and a flag may be set so as not to trigger an alarm. While a specific example with AND gates has been shown the data can be combined in may ways with known gates for example NAND, NOR, OR, NOT, XOR, XNOR gates. In some embodiments, the gated logic may be embodied in a truth table.

While the exemplary embodiments have been described in some detail, by way of example and for clarity of understanding, those of skill in the art will recognize that a variety of modifications, adaptations, and changes may be employed. Hence, the scope of the present invention should be limited solely by the appended claims.

While the exemplary embodiments have been described in some detail, by way of example and for clarity of understanding, those of skill in the art will recognize that a variety of modifications, adaptations, and changes may be employed. Hence, the scope of the present invention should be limited solely by the appended claims.

What is claimed is:

1. A system for monitoring a physiological status of a person, the system comprising:
a person measuring system comprising,
a plurality of sensors, each sensor configured to couple to the person and provide a sensor output, and
a wireless communication device coupled to the plurality of sensors and configured to transfer data from the plurality of sensors; and
a remote monitoring system coupled to the wireless communication device and configured to receive the data from the plurality of sensors, wherein the remote monitoring system is positioned remote from the person;
wherein at least one of the person measuring system or the remote monitoring system is configured to monitor the physiological status of the person and to determine a distress of the person in response to a combination of the sensor outputs.

2. The system of claim 1, wherein the person measuring system is configured to determine the distress.

3. The system of claim 1, wherein the remote system is configured to determine the distress.

4. The system of claim 1, wherein the person measuring system and the remote system are configured to determine the distress.

5. The system of claim 2, wherein each sensor comprises circuitry configured to provide the output and wherein the person monitoring system comprises an adherent patch configured to adhere to the person and support the plurality of sensors and the circuitry.

6. The system of claim 1, wherein the plurality of sensors comprises a combination of sensors configured to measure at least two of bioimpedance, heart rate, a heart rhythm, HRV, HRT, heart sounds, respiration rate, respiratory rate variability, respiratory sounds, blood pressure, activity, posture, wake/sleep, SpO2, orthopnea, temperature, heat flux or an accelerometer.

7. The system of claim 1, wherein the wireless communication device is configured to receive instructional data from the remote monitoring system.

8. The system of claim 1, further comprising a processor system coupled to the plurality of sensors and to the wireless communication device, the processor system configured to receive data from the plurality of sensors and generate processed monitored individual data.

9. The system of claim 8, wherein the processor system comprises at least one processor located with the remote monitoring system.

10. The system of claim 8 wherein the person measuring system comprises a monitoring unit and wherein the processor system comprises a processor located with the monitoring unit.

11. The system of claim 10, wherein the monitoring unit comprises logic resources configured to determine the distress of the person and detect a negative physiological event of the person.

12. The system of claim 1, the remote monitoring system comprises logic resources configured to determine the distress of the person and to detect a negative physiological event of the person.

13. The system of claim 1, wherein the person measuring system comprising the plurality of sensors is configured to provided initiation, programming, measuring, storing, analyzing and communicating data of the monitored person and wherein the remote monitoring system is configured to predict and display a physiological event of the monitored person.

14. The system of claim 1, wherein the plurality of sensors comprises at least one of bioimpedance, heart rate, heart rhythm, HRV, HRT, heart sounds, respiration rate, respiratory rate variability, respiratory sounds, blood pressure, activity, posture, wake/sleep, SpO2, orthopnea, temperature, heat flux or accelerometer.

15. The system of claim 14, wherein the plurality of sensors is configured to measure activity with an activity sensor comprising at least one of a ball switch, an accelerometer, a minute ventilation sensor, a heart rate sensor, a bioimpedance noise sensor, a skin temperature sensor, a heat flux sensor, a blood pressure sensor, a muscle noise sensor or a posture sensor.

16. The system of claim 1, wherein the plurality of sensors is configured to switch from a first mode to a second mode, the first mode different from the second mode.

17. The system of claim 16, wherein the first mode and the second mode comprise at least one of a stand alone mode for communication directly with the remote monitoring system, a communication mode for communication an implanted device, a mode for communication with a single implanted device, a mode to coordinate different devices coupled to the plurality of sensors with different device communication protocols.

18. The system of claim 17, wherein the person measuring system comprising the plurality of sensors is configured to deactivate selected sensors to reduce redundancy.

19. The system of claim 1, wherein the distress of the monitored person is determined in response to a weighted combination change in sensor outputs.

20. The system of claim 1, further comprising a processor system and wherein the processor system is configured to determine distress of the monitored individual and detect a physiological event when a rate of change of at least two sensor outputs comprises an abrupt change in the sensor outputs as compared to a change in the sensor outputs over a longer period of time.

21. The system of claim 1, further comprising a processor system and wherein the processor system is configured to determine distress of the person and a physiological event in response to a tiered combination of at least a first sensor output and a second sensor output, wherein the processor system is configured to verify the first sensor output with at least a second sensor output when the first sensor output indicates the that physiological event comprises a problem for the person.

22. The system of claim 1, wherein the remote monitoring system is configured to determine the distress of the monitored person in response to a variance from baseline values of sensor outputs.

23. The system of claim 22, wherein the baseline values are defined by a look up table.

24. The system of claim 1, further comprising a processor system and wherein the plurality of sensors comprises at least a first sensor having a first sensor output, a second sensor having a second sensor output and a third sensor having a third sensor output, the processor system configured to combine output of each of the first sensor, the second sensor and the third sensor to determine the distress of the person.

25. The system of claim 24, wherein the processor system is configured to determine the distress of the person in response to the first sensor output at a first high value greater than a baseline value and at least one of the second sensor output or the third sensor outputs at a second high value also sufficiently greater than a second baseline value to indicate the distress of the person.

26. The system of claim 24, wherein the processor system is configured to determine the distress of the person in response to time weighting the output of each of the first sensor, the second and third sensor, such that the time weighting indicates a recent event that is indicative of the distress of the monitored person.

27. The system of claim 1, further comprising a processor system and wherein the processor system is configured to track the person's physiological status with the plurality of sensors and detect and predict negative physiological events.

28. The system of claim 1, wherein the outputs of the plurality of sensors comprise multiple sensing vectors that include redundant vectors.

29. The system of claim 1, wherein the plurality of sensors comprises current delivery electrodes and sensing electrodes.

30. The system of claim 1, further comprising a processor system comprising a tangible medium and wherein the processor system is coupled to outputs of the plurality of sensors and configured to calculate blended indices to monitor the person.

31. The system of claim 30, wherein the blended indices comprise at least one of heart rate, respiratory rate, response to activity, heart rate divided by respiratory rate response to posture change, heart rate plus respiratory rate, heart rate divided by respiratory rate plus bioimpedance, or minute ventilation and accelerometer.

32. The system of claim 1, wherein the person measuring system is configured to cycle data sampling among each sensor of the plurality of sensors to minimize energy consumption of the plurality of sensors.

33. The system of claim 32, wherein the person measuring system is configured to sample data at different times for each sensor of the plurality.

34. The system of claim 1, wherein the plurality of sensors comprises a first sensor and a second sensor and wherein the first sensor comprises a core sensor configured to continuously monitor and determine the distress and wherein the person measuring system is configured to verify distress with the second sensor in response to the core sensor raising a flag.

35. The system of claim 1, wherein at least a first portion of the sensors are used for short term tracking, and at least a second portion of the sensors are used for long term tracking, the second portion different from the first portion.
